(19) United States
(12) Patent Application Publication
(10) Pub. No.: US 2025/0065024 A1
(43) Pub. Date: Feb. 27, 2025

(54) PROVIDING TREATMENT FLUID FOR DIALYSIS THERAPY

(71) Applicant: GAMBRO LUNDIA AB, Lund (SE)

(72) Inventors: Vishnu SHYAM, Lund (SE); Christian VARTIA, Veberöd (SE)

(21) Appl. No.: 18/721,011

(22) PCT Filed: Dec. 20, 2022
(86) PCT No.: PCT/EP2022/086926

§ 371 Date: Jun. 17, 2024

(30) Foreign Application Priority Data
Dec. 22, 2021 (SE) 2151585-3

(51) Int. Cl. A61M 1/16

(52) U.S. Cl. CPC A61M 1/1656; A61M 1/1601

(57) ABSTRACT

A system for providing treatment fluid for dialysis therapy comprises a fluid generation unit, which is configured to generate the treatment fluid by mixing water with one or more substances, and a dehumidifier unit, which is configured to receive a first stream of air, process the first stream of air for extraction of liquid water, and provide the liquid water for the fluid generation unit. The system further comprises a humidifier unit, which is configured to receive a second stream of air, process the second stream of air to generate a third stream of air with increased humidity compared to the second stream of air. The humidifier unit is arranged to include at least part of the third stream of air in the first stream of air. Depending on configuration, the system is capable of reducing the need for tap water, facilitate installation, facilitate purification and/or improve indoor environment.

### TECHNICAL FIELD

The present disclosure relates generally to dialysis therapy, and in particular to a technique of producing dialysis fluid for use in dialysis therapy.

### BACKGROUND ART

In the treatment of individuals suffering from acute or chronic renal insufficiency, dialysis therapy may be needed.

One category of dialysis therapy is extracorporeal (EC) blood therapy, in which blood from a patient is pumped through an EC blood circuit back to the patient. A blood filtration unit, commonly known as a dialyzer, is arranged in the EC blood circuit to interface the blood by a semi-permeable membrane. There are various types of EC blood therapy, including hemodialysis (HD), hemofiltration (HF), and hemodiafiltration (HDF). In HD, a treatment fluid (“dialysis fluid”) is pumped through the dialyzer. Water and substances are exchanged between the treatment fluid and the blood through the semi-permeable membrane, primarily driven by a diffusive gradient across the semi-permeable membrane. In HF, treatment fluid is not pumped through the dialyzer. Instead, a treatment fluid (“replacement fluid”) is infused into the blood in the EC blood circuit during therapy, upstream and/or downstream of the dialyzer, and fluid and substances are transported from the blood through the semi-permeable membrane, primarily by convection. In HDF, a dialysis fluid is pumped through the dialyzer, similar to HD, and a replacement fluid is infused into the blood in the EC blood circuit, similar to HF.

Another category of dialysis therapy is peritoneal dialysis (PD). In PD therapy, a treatment fluid (“dialysis fluid”) is infused into the individual's peritoneal cavity. This cavity is lined by a peritoneal membrane (“peritoneum”) which is highly vascularized. Substances are removed from the patient's blood mainly by diffusion across the peritoneum into the dialysis fluid. Excess fluid (water) is removed by osmosis induced by the dialysis fluid being hypertonic. Used or spent dialysis fluid is drained from the patient, removing waste, toxins and excess water from the patient. This cycle may be repeated, for example multiple times. There are various types of PD therapies, including continuous ambulatory PD (CAPD), automated PD (APD), tidal PD (TPD), and continuous flow PD (CFPD). CAPD is a manual dialysis treatment, in which the flow of treatment fluid into and out of the patient is driven by gravity. APD is performed by a dialysis machine, commonly known as a cycler, which is fluidly connected to the peritoneal cavity and operated to automatically transfer treatment fluid to and from the peritoneal cavity in accordance with a predefined schedule, for example during the night while the patient is sleeping.

Conventionally, treatment fluid for PD is delivered in pre-filled bags to the point-of-care, for example an intensive care unit or the home of the patient. EC blood therapy may also use pre-filled bags of treatment fluid, for example in an intensive care unit, for treatment of acute kidney failure. Treatment fluid for treatment of chronic kidney failure by EC blood therapy is typically produced by the dialysis machine itself, by mixing one or more concentrates with purified water. Recently, dialysis machines that produce treatment fluid for PD therapy have also been proposed.

Local production of treatment fluid at the point-of-care is attractive since it reduces the cost and environmental impact of transporting large amounts of ready-made treatment fluid and the burden of storing and handling pre-filled bags. However, production of treatment fluid requires access to purified water. Typically, a water purification unit is connected to a tap water source, and a fluid generation unit is operated to mix one or more concentrates with the purified water to generate the treatment fluid. Large amounts of tap water may be consumed. In PD therapy, about 15 liters of treatment fluid may be consumed daily. In EC blood therapy, more than 100 liters of treatment fluid may be consumed during a single treatment session.

To improve the patient's quality of life, and also to reduce the cost of treatment, dialysis may be performed locally in the home of the patient (“home dialysis”). As noted, local production of treatment fluid requires access to tap water, and preferably also to a drain for disposal of spent treatment fluid. Installation in the home of the patient may thus involve plumbing work to install tubing between a sanitary area and the room where treatment is performed, for example a bedroom. The plumbing work adds to the cost of treatment, and extended tubing increases the risk of leaks and consequential water damage.

Another obstacle to home dialysis is the need to purify the tap water. A water purification unit has to be installed in the patient's home, as a separate machine or included in the dialysis machine. The water purification unit is configured to process the tap water to ensure that the treatment fluid complies with quality standards, for example in terms of the content of various ions, molecules and larger particles as well as microorganisms. For example, the water purification unit may be configured to perform reverse osmosis, which increases the cost of the dialysis system as a whole.

U.S. Pat. No. 10,632,242 discloses a dialysis machine that is designed to operate in areas where resources such as energy and clean water are scarce. The dialysis machine comprises a condenser-based water generator which is powered by a solar panel to extract water from ambient air. After passing an ultrafilter, the extracted water may be mixed with concentrates to form a dialysis fluid or be used for generating saline to be infused into the patient's blood. The dialysis fluid is added as a supplement to regenerated dialysis fluid, which is produced from spent dialysis fluid by use of a sorbent device. The amount of extracted water is sufficient to produce a supplement to regenerated dialysis fluid. However, when operated indoors, the available volume of air is limited and the proposed dialysis machine is generally unable to extract water at the quantities needed to generate the PD fluid that is required for daily PD treatment of a patient. Further, extracting significant quantities of water from air in an indoor environment may lower the humidity to an extent that is uncomfortable or even unhealthy for the patient.

### SUMMARY

It is an objective to at least partly overcome one or more limitations of the prior art.

One objective is to provide a technique that facilitates treatment by dialysis therapy in an indoor environment, for example in the home of a patient.

A further objective is to provide cost-efficient system for providing treatment fluid for dialysis therapy.

One or more of these objectives, as well as further objectives that may appear from the description below, are at least partly achieved by a system for providing treatment fluid for dialysis therapy, and related methods, embodiments thereof being defined by the dependent claims.

A first aspect is a system for providing treatment fluid for dialysis therapy. The system comprises: a fluid generation unit configured to generate the treatment fluid by mixing water with one or more substances; a dehumidifier unit configured to receive a first stream of air, process the first stream of air for extraction of liquid water, and provide the liquid water for the fluid generation unit; and a humidifier unit configured to receive a second stream of air, process the second stream of air to generate a third stream of air with increased humidity compared to the second stream of air, wherein the humidifier unit is arranged to include at least part of the third stream of air in the first stream of air.

The system of the first aspect combines a dehumidifier unit with a humidifier unit that is configured to generate a humid air stream by use of tap water. The humidifier unit is thereby capable of increasing the available amount of water to be extracted by the dehumidifier unit from a given air volume. This makes the system suitable for use in an indoor environment where the available air volume may be limited. The provision of the humidifier unit also enables an acceptable air humidity to be maintained in such an indoor environment even if the dehumidifier unit should be operated to extract large quantities of water from the air. Even if tap water is required for the operation of the humidifier unit, the total amount of tap water required by the system to generate a given volume of treatment fluid may be reduced if the dehumidifier unit is operated to extract water in excess of the water that is added into the humid air stream by the humidifier unit. By configuring the system to require a reduced amount of tap water, the need for a fixed installation of tubing is mitigated since the tap water may, for example, without difficulty be manually transferred from a fixed outlet of tap water to a tank of the system.

The system opens up several possibilities to facilitate installation. In some embodiments, the humidifier unit and the dehumidifier unit are physically separated from each other, allowing them to be independently arranged within the indoor environment. For example, the humidifier unit may be located close to a fixed outlet of tap water, and the dehumidifier unit may be located close to the fluid generation unit. The transport of water from the humidifier unit to the dehumidifier unit is performed by the humid air stream that is generated by the humidifier unit. The humid air stream may be conducted in a tubing from the humidifier unit to the dehumidifier unit. Such tubing is simple to install and, since it conducts gas (air) rather than liquid water, does not significantly increase the risk of water damage. In an alternative, no tubing is used for the air transport, and the humidifier and dehumidifiers units are thus fully decoupled from each other. Instead, the humid air stream is released by the humidifier unit into its immediate surroundings, and the incoming air stream to be dehumidified is received by the dehumidifier unit from its immediate surroundings. Over time, as the system is operated, at least part of the humid air that is emitted by the humidifier unit will be transported to the dehumidifier unit, for example by natural and/or forced convection within the indoor environment. The absence of tubing between the humidifier and dehumidifier units will increase the freedom to select a convenient placement of the respective unit. In some embodiments, the humidifier and dehumidifier units are instead combined into a unitary structure and are thus co-located. The transport of the humid air stream from the humidifier unit to the dehumidifier unit may be performed in any suitable way internally of the unitary structure. The unitary combination results in a single unit which may be made compact and suitable for installation in a confined space such as a home. Further, system control may be facilitated in a system with a unitary combination of humidifier and dehumidifier units compared to a system with physically separated humidifier and dehumidifier units. In some embodiments, the dehumidifier unit comprises a desiccant which is arranged to adsorb and/or absorb water in the incoming air stream. The desiccant may be in liquid or solid form. The use of a desiccant for dehumidification enables water to be extracted in a power-efficient way. Further, compared to condenser-based dehumidification, water may be efficiently extracted from air that holds smaller amounts of moisture. In some embodiments, by proper choice of desiccant, the dehumidification unit performs an inherent purification of the extracted water. The purification is achieved by ensuring that the desiccant has a high selectivity for water and thus preferentially adsorbs and/or absorbs water molecules compared to other molecules, as well as ions and particles, present in the incoming gas stream. Such a dehumidification unit has the potential of replacing or supplementing a water treatment unit that may be required for conventional generation of treatment fluid. For example, water treatment by reverse osmosis may be obviated, resulting in cost savings.

In some embodiments, the dehumidifier unit comprises a desiccant, which is arranged to adsorb and/or absorb moisture from the first stream of air and which is processed by the dehumidification unit to extract the liquid water from the desiccant.

In some embodiments, the desiccant is configured to have a high selectivity towards water.

In some embodiments, the liquid water that is extracted from the first stream of air has conductivity of less than 10 μS/cm, and preferably less than 5 μS/cm or 1 μS/cm.

In some embodiments, the system further comprises a control arrangement, which is configured to jointly operate the humidifier unit and dehumidifier unit to achieve a set point that defines air humidity within a space that comprises the humidifier unit and the dehumidifier unit.

In some embodiments, the control arrangement is further configured to operate the dehumidifier unit to achieve a set point that defines an amount of liquid water extracted per unit time.

In some embodiments, the control arrangement is further configured to deactivate the humidifier unit and dehumidifier unit when an accumulated amount of liquid water exceeds a threshold value.

In some embodiments, the system further comprises at least one humidity sensor, which is arranged to generate a measurement signal representing the air humidity within the space.

In some embodiments, the control arrangement is configured to perform a step-response test by causing a step-change in operating performance of at least one of the humidifier unit or the dehumidifier unit, monitor the measurement signal, and determine control parameter values for the control arrangement based on the measured air humidity within the space as a function of time subsequent to the step-change.

In some embodiments, the at least one humidity sensor is arranged to measure the air humidity in at least one of the first and second streams of air.

In some embodiments, the system further comprises a connection sensor, which is configured to sense a fluid connection state between the humidifier unit and the dehumidifier unit, and the control arrangement is operable to modify its operation in correspondence with the fluid connection state sensed by the connection sensor.

In some embodiments, the system further comprises at least one fluid connecting device between the humidifier unit and the dehumidifier unit, and the humidifier unit is configured to emit at least part of the third stream of air into the at least one fluid connecting device and the dehumidifier unit is configured to receive the first stream of air from the at least one fluid connecting device.

In some embodiments, the humidifier unit and the dehumidifier unit are combined into a unitary structure.

In some embodiments, the dehumidifier unit is configured to output a fourth stream of air which is generated when the first stream of air is processed for extraction of the liquid water, and the system further defines a fluid channel, which is configured to re-direct at least part of the fourth stream of air to an air inlet of the humidifier unit, to include said at least part of the fourth stream of air in the first stream of air.

In some embodiments, the fluid channel is arranged to redirect the fourth stream of air to the air inlet of the humidifier unit, so that the fourth stream of air forms the first stream of air.

In some embodiments, the system comprises a flow controller which is operable to set how much of the fourth stream of air that is re-directed to the air inlet of the humidifier unit.

In some embodiments, the system is switchable, by operation of the flow controller, between a first operating mode and a second operating mode, wherein the system, in first operating mode, is configured to admit surrounding air into the second stream of air and provide at least part of a fourth stream of air into the surrounding air, and wherein the system, in the second operating mode, is configured to direct the fourth stream of air to the air inlet of the humidifier unit, so that the fourth stream of air forms the first stream of air.

In some embodiments, the system, in the first operating mode, is configured to provide all of the fourth stream of air into the surrounding air.

In some embodiments, the system comprises an inlet line, which is connected to the air inlet of the humidifier unit, and an outlet line, which is connected to an outlet of the dehumidifier unit to receive the fourth stream of air from the dehumidifier unit, wherein the fluid channel is fluidly connected at a first junction on the outlet line and at a second junction on said the inlet line, and wherein the flow controller is arranged to control an air flow rate through the fluid channel from the outlet line to the inlet line.

In some embodiments, the humidifier unit is configured to emit the third stream of air into surrounding air, and the dehumidifier unit is configured to receive the first stream of air from the surrounding air.

In some embodiments, the humidifier unit is physically separated from the dehumidifier unit.

In some embodiments, the humidifier unit is configured to receive tap water and process the tap water to generate the third stream of air from the second stream of air and the tap water.

In some embodiments, the humidifier unit is connected to receive the tap water from a tank, wherein the system is configured to estimate a time point of depletion of the tank, based on a signal from a level sensor of the tank or based on measured air humidity within a space that comprises the humidifier unit and the dehumidifier unit, and output, in advance of said time point, a user instruction to add tap water to the tank.

In some embodiments, the system is configured to estimate the time point of depletion by use of a calculation model, which is configured to predict the extraction of the liquid water as a function of air humidity, and by further use of data indicative of consumption of the treatment fluid by an operating therapy system, which is connected to receive the treatment fluid from the system.

In some embodiments, the system further comprises a storage unit, which is arranged to receive and accumulate the liquid water from the dehumidifier unit or the treatment fluid from the fluid generation unit.

A second aspect is a method of providing treatment fluid for use in dialysis therapy. The method comprises: processing a first stream of air for extraction of liquid water; generating the treatment fluid by mixing at least part of the liquid water with one or more substances; processing a second stream of air to generate a third stream of air with increased humidity compared to the second stream of air; and including at least part of the third stream of air in the first stream of air.

A third aspect is a computer-implemented method of operating the system of the first aspect or any of its embodiments. The method comprises: receiving at least one measurement signal representing the air humidity within a space that comprises the humidifier unit and the dehumidifier unit, and jointly operating the humidifier unit and dehumidifier unit to achieve a predefined air humidity within the space as indicated by the at least one measurement signal.

The embodiments of the first aspect may be adapted as embodiments of the second or third aspect.

A fourth aspect is a computer-readable medium comprising program instructions, which when executed by a processor causes the processor to perform the method of the second or third aspect, or any embodiment thereof. The computer-readable medium may be a non-transitory medium or a propagating signal.

Still other objectives, aspects, embodiments and technical effects, as well as features and advantages may appear from the following detailed description, from the attached claims as well as from the drawings.

### BRIEF DESCRIPTION OF THE DRAWINGS

Embodiments will now be described in more detail with reference to the accompanying and schematic drawings.

FIGS. 1A-1B are overviews of example dialysis systems for peritoneal dialysis and extracorporeal blood treatment, FIG. 1C is a more detailed block diagram of an example dialysis system comprising a combination of a humidifier unit and a dehumidifier unit, and FIGS. 1D-1F are block diagrams of an example fluid generation unit, an example humidifier unit and an example dehumidifier unit for use in the dialysis systems of FIGS. 1A-1B.

FIG. 2A is a side view of a non-connected combination of humidifier and dehumidifier units, and FIG. 2B shows a plan view of a home environment comprising installation examples of the non-connected combination.

FIG. 3 is flow chart of an example method of installing and operating a combination of humidifier and dehumidifier units.

FIG. 4 shows a plan view of a home environment comprising an installation example of a connected combination of humidifier and dehumidifier units.

FIGS. 5A-5D are block diagrams of the connected combination of humidifier and dehumidifier units in accordance with various implementation examples.

FIG. 6 is a flow chart of a method of installing and operating the combination in FIG. 5C.

FIGS. 7A-7D illustrate humidifiers and dehumidifiers that are operable in any one of the non-connected and connected combinations.

FIG. 8 is a flow chart of a method of providing treatment fluid.

### LIST OF ABBREVIATIONS

APD Automated peritoneal dialysis
CAPD Continuous ambulatory peritoneal dialysis
CPD Continuous flow peritoneal dialysis
DHU Dehumidifier unit
DIA Incoming air stream (dehumidifier unit)
DOA Outgoing air stream (dehumidifier unit)
EC extracorporeal
EW Extracted liquid water
FCD Fluid connecting device
FGU Fluid generation unit
FPS Fluid preparation system
Ha Ambient humidity
HD Hemodialysis
Hdi Inlet humidity (dehumidifier unit)
HDF Hemodiafiltration
Hdo Outlet humidity (dehumidifier unit)
HF Hemofiltration
Hhi Inlet humidity (humidifier unit)
Hho Outlet humidity (humidifier unit)
HIA Incoming air stream (humidifier unit)
HOA Outgoing air stream (humidifier unit)
HU Humidifier unit
MOF Metal-organic framework
PC Peritoneal cavity
PD peritoneal dialysis
RH Relative humidity
RO Reverse osmosis
SBU Secondary building unit
TPD Tidal peritoneal dialysis
TW Tap water

### DETAILED DESCRIPTION OF EXAMPLE EMBODIMENTS

Embodiments will now be described more fully hereinafter with reference to the accompanying drawings, in which some, but not all, embodiments are shown. Indeed, the subject of the present disclosure may be embodied in many different forms and should not be construed as limited to the embodiments set forth herein; rather, these embodiments are provided so that this disclosure may satisfy applicable legal requirements.

Also, it will be understood that, where possible, any of the advantages, features, functions, devices, and/or operational aspects of any of the embodiments described and/or contemplated herein may be included in any of the other embodiments described and/or contemplated herein, and/or vice versa. In addition, where possible, any terms expressed in the singular form herein are meant to also include the plural form and/or vice versa, unless explicitly stated otherwise. As used herein, “at least one” shall mean “one or more” and these phrases are intended to be interchangeable. Accordingly, the terms “a” and/or “an” shall mean “at least one” or “one or more”, even though the phrase “one or more” or “at least one” is also used herein. As used herein, except where the context requires otherwise owing to express language or necessary implication, the word “comprise” or variations such as “comprises” or “comprising” is used in an inclusive sense, that is, to specify the presence of the stated features but not to preclude the presence or addition of further features in various embodiments.

As used herein, the terms “multiple”, “plural” and “plurality” are intended to imply provision of two or more elements, whereas the term a “set” of elements is intended to imply a provision of one or more elements. The term “and/or” includes any and all combinations of one or more of the associated listed elements.

It will furthermore be understood that although the terms first, second, etc. may be used herein to describe various elements, these elements should not be limited by these terms. These terms are only used to distinguish one element from another. For example, a first element could be termed a second element, and, similarly, a second element could be termed a first element, without departing the scope of the present disclosure.

Well-known functions or constructions may not be described in detail for brevity and/or clarity. Unless otherwise defined, all terms (including technical and scientific terms) used herein have the same meaning as commonly understood by one of ordinary skill in the art to which this disclosure belongs.

As used herein, “dialysis therapy” refers to any therapy that replaces or supplements the renal function of a patient by use of treatment fluid. Dialysis therapy includes, without limitation, extracorporeal blood therapy and peritoneal dialysis therapy.

As used herein, “treatment fluid” refers to any fluid that is consumed as a result of dialysis therapy. Treatment fluid includes, without limitation, dialysis fluid, replacement fluid and substitution fluid.

As used herein, “ambient air” or “open air” refers to air located within a confined space, such as an apartment, a room, or the like but outside of the system described herein. When referring to an air stream being received from the surroundings, the air stream is taken from ambient air. Similarly, when referring to an air stream being emitted into the surroundings, the air stream is included in ambient air.

Like reference signs refer to like elements throughout.

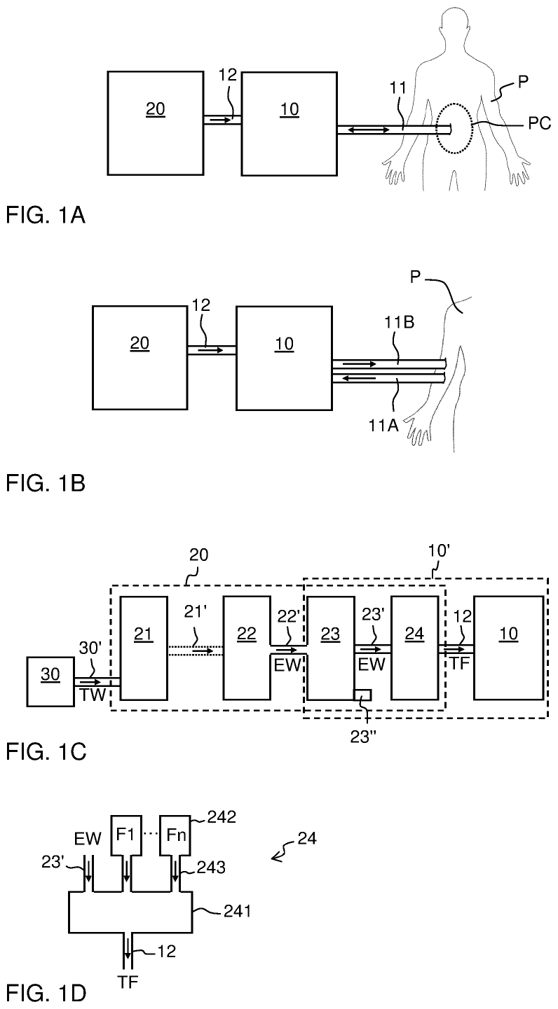

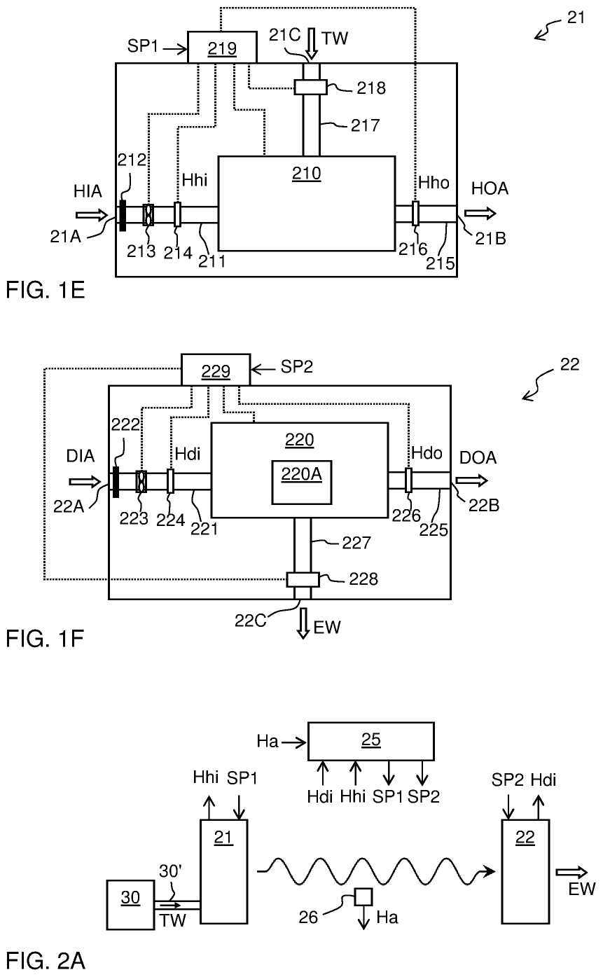

The present disclosure relates to a technique of generating treatment fluid for a dialysis system. The technique is applicable to both peritoneal dialysis (PD) therapy or extracorporeal (EC) blood therapy. For context only, fluid generation in relation to PD therapy and EC blood therapy will be briefly discussed with reference to FIGS. 1A-1B.

FIG. 1A is a generic overview of a dialysis system for PD therapy. The dialysis system comprises a therapy system 10, which is fluidly connected to the peritoneal cavity PC of a patient P. As indicated by a double-ended arrow, the therapy system 10 is operable to convey fresh treatment fluid into the peritoneal cavity PC and to receive spent treatment fluid from the peritoneal cavity on a fluid path 11. The fluid path 11 may be defined by tubing that connects to an implanted catheter (not shown) in fluid communication with the peritoneal cavity PC. The therapy system 10 may be configured for any type of PD therapy. In one example, the therapy system 10 comprises one or more containers that are manually handled to perform CAPD. In another example, the therapy system 10 comprises a dialysis machine (“cycler”) that performs the dialysis therapy. The dialysis system further comprises a fluid preparation system, FPS, 20, which is configured to generate treatment fluid for use by the therapy system 10. The treatment fluid is supplied from the FPS 20 to the therapy system 10 on a fluid path 12. The spent treatment fluid may be handled by the therapy system 10, or transferred for handling by the FPS 20. The spent treatment fluid may be stored, regenerated or sent to drain, or any combination thereof.

FIG. 1B is a generic overview of a dialysis system for EC blood therapy. The dialysis system comprises a therapy system 10, which is fluidly connected to the vascular system of a patient P on a fluid path. In the illustrated example, the fluid path is defined by tubing 11A for blood extraction and tubing 11B for blood return. As indicated by arrows, the therapy system 10 is operable to draw blood from the patient P through tubing 11A, process the blood, and return the processed blood to the patient through tubing 11B. The tubing 11A, 11B is connected to an access device (for example a catheter, graph or fistula, not shown) in fluid communication with the vascular system of the patient P. The therapy system 10 may be configured to process the blood by any form of EC blood therapy, such as HD, HF or HDF. In such therapy, treatment fluid is consumed. The treatment fluid is supplied from the FPS 20 to the therapy system 10 on the fluid path 12. The spent treatment fluid may be handled by the therapy system 10 or transferred for handling by the FPS 20. The spent treatment fluid may be stored, regenerated or sent to drain, or any combination thereof.

FIG. 1C depicts a dialysis system in more detail, in particular the FPS 20. In accordance with some embodiments, the FPS 20 comprises a humidifier unit, HU, 21 and a dehumidifier unit, DHU 22. The HU 21 is arranged to receive tap water, TW, on a fluid path 30′ from a tap water source 30. The tap water source 30 may be a permanent outlet (water tap, faucet, spigot, etc.) or a tank, which is manually or automatically replenished with tap water as needed. The HU 21 is configured to generate, based on TW, a humidified air stream. The DHU 22 is configured to extract liquid water, EW, from an incoming air stream. As will be described in detail below, a fluid path 21′ is established between the HU 21 and the DHU 22, so that at least part of the humified air stream is included in the incoming air stream to the DHU 22. Thereby, at least part of EW as extracted by the DHU 22 corresponds to TW as received and processed by the HU 21. In some embodiments, the fluid path 21′ comprises one or more confined (physical) channels and is defined by a connection device that extends between the HU 21 and the DHU 22. In other embodiments, the fluid path 21′ is established by the HU 21 emitting the humified air stream into the surrounding air.

In the example of FIG. 1C, EW is transferred from the DHU 22 to a storage unit 23 on a fluid path 22′. The storage unit 23 is a tank, container or the like for intermediate storage of EW. A fluid path 23′ extends from the storage unit 23 to a fluid generation unit, FGU, 24. The FGU 24 is configured to prepare treatment fluid, TF, by mixing EW with one or more substances, for example in the form of one or more concentrates. The TF is then supplied to the therapy system 10 on fluid path 12. The storage unit 23 allows the production rate of EW to be decoupled from the production rate of TF, which may simplify the FGU 24. Furthermore, the storage unit 23 allows the system to use a portion of EW for cleaning the therapy system 10 and/or the FPS 20. For example, a certain daily reject water volume may be required to counteract scaling in the HU 21. In an alternative, the storage unit 23 or an additional storage unit (not shown) is arranged to receive TF from the FGU 24, and TF is supplied to the therapy system 10 from the storage unit 23 or the additional storage unit. In a further alternative, the storage unit 23 is omitted.

The partitioning into an FPS 20 and a therapy system 10 is arbitrary and made only for explanatory purposes. FIG. 1C indicates an alternative partitioning, which will be used in some examples below (FIGS. 2B and 4). In this alternative partitioning, the dialysis system comprises the HU 21, the DHU 22 and a compound therapy system 10′, which includes the therapy system 10, the FGU 24 and the storage unit 23 (if present). In practice, the HU 21, the DHU 22, the storage unit 23, the FGU 24 and the therapy system 10 may be separate devices or be structurally combined in any suitable way.

FIG. 1D is a schematic view of the FGU 24. FIG. 1D is merely intended to illustrate the overall function of the FGU 24 and is not intended to be limiting in any way. In the illustrated example, the FGU 24 is arranged to receive EW from the storage unit 23 on fluid path 23′. If the storage unit 23 is omitted, the FGU 24 receives EW from the DHU 22 on fluid path 22′. The FGU 24 comprises a mixing section 241, which is configured to mix EW with one or more concentrates, represented as F1, . . . , Fn in FIG. 1D. The respective concentrate F1, . . . , Fn may be in liquid or powdery format. The concentrate is held in a reservoir 242 and supplied to the mixing structure 241 on a path 243. The EW is mixed with the one or more concentrates F1, . . . , Fn in the mixing structure 241 to form TF, which is supplied to the therapy system 10 on fluid path 12. The mixing structure 241 may be of any conventional configuration. It is to be understood that the FGU 24 may comprise additional equipment, such as devices for dosing the concentrate(s) and EW, a device for mixing, a heating device, a degassing device, pumps, sensors, etc. Any available concentrate that is conventionally used for generating treatment fluid for PD therapy or EC blood therapy by mixing with water may be used. The FGU 24 may be configured to produce TF in batches or on-demand. In on-demand production, the production rate of TF is adapted or matched to the consumption rate of TF by the therapy system 10.

In some embodiments, TF for use in EC blood therapy on patients with chronic kidney disease (CKG) is generated by mixing a single concentrate with water at a dilution ratio of 10-50 by volume. In a non-limiting example, the single concentrate comprises lactate, sodium, potassium, calcium, magnesium, glucose and chloride. Such a concentrate is, for example, commercially available for the PureFlow SL system from NxStage. Alternatively, TF may be generated by mixing two concentrates with water. For example, a base concentrate and an acid concentrate may be mixed with water at a dilution ratio of 10-50. Such concentrates are commercially available and well-known in the art. In a non-limiting example, the base concentrate comprises a buffer, for example bicarbonate, and the acid concentrate comprises sodium, potassium, calcium, magnesium, glucose, acetate and chloride. In some acid concentrates, acetate is replaced or supplemented by another acid, for example citric acid. In some embodiments, TF for CRRT treatment of patients with acute kidney injury (AKI) is generated by mixing at least one concentrate with water. In a non-limiting example, such a dialysis/replacement fluid comprises bicarbonate, sodium, potassium, calcium, magnesium, phosphate, glucose, acetate and chloride. In one example, a base concentrate and an electrolyte concentrate may be mixed with water to form the dialysis/replacement fluid. For example, the base concentrate may be an alkaline hydrogen carbonate solution, and the electrolyte concentrate may be an acidic glucose-based electrolyte solution.

TF for use in PD therapy may be generated by mixing at least one concentrate with water. Example compositions of concentrates to be mixed with water are disclosed in US2018/0021501 and WO2017/193069, which are incorporated herein by reference. In one example, the one or more concentrates comprises ions and/or salts, such as lactate, acetate, citrate, bicarbonate, KCl, MgCL2, CaCl2, NaCl, and an osmotic agent. In any of the embodiments described herein, the osmotic agent may be, or include, glucose (or polyglucose), L-carnitine, glycerol, icodextrin, or any other suitable agent. For example, icodextrin is a glucose polymer preparation commonly used as osmotic agent in PD fluids. Alternative osmotic agents may be fructose, sorbitol, mannitol and xylitol. It is noted that glucose is also sometimes named as dextrose in the PD field. The term glucose is herewith intended to comprise dextrose.

FIG. 1E is a block diagram of an example humidifier unit, HU, 21. An evaporation unit 210 is configured to evaporate (vaporize) TW for generation gaseous moisture and add at least part of the gaseous moisture to an incoming air stream HIA. Thereby, an outgoing air stream HOA with increased humidity is generated. The evaporation unit 210 may be of conventional design. In some embodiments, the evaporation unit 210 is configured to process the TW by applying heat, optionally at low pressure, to generate the gaseous moisture. The HU 21 in FIG. 1E comprises an air inlet 21A, an air outlet 21B, and a tap water inlet 21C. The air inlet 21A opens to an air inlet channel 211, which extends to the evaporation unit 210. An air filter 212 is arranged in the air inlet channel 211 for removal of particulate matter, such as debris, dust, etc. A pumping device 213 is arranged downstream of the air filter 212, to generate and drive HIA through the evaporation unit 210. A humidity sensor 214 is arranged in the air inlet channel 211 to sense the inlet humidity Hhi of HIA. The air outlet 21B opens to an air outlet channel 215, which extends from the evaporation unit 210. A humidity sensor 216 is arranged in the air outlet channel 215 to sense the outlet humidity Hho of HOA. The tap water inlet 21C opens to a water inlet channel 217 which extends to the evaporation unit 210. A flow controller 218 is arranged in the water inlet channel 217 to control the flow of TW into the evaporation unit 210. The flow controller 218 may for example comprise a valve and/or a pumping device. A local control unit 219 is configured to generate control signals for the pumping device 213, the flow controller 218, and the evaporation unit 210, based on sensor data (“measurement signals”) from the humidity sensors 214, 216, to achieve set point data SP1. For example, the set point data SP1 may include a target value for the outlet humidity Hho and/or a target value for the flow rate of HOA. The local control unit 219 may, for example, be implemented as a P, PI or PID controller.

FIG. 1F is a block diagram of an example dehumidifier unit, DHU, 22. A water extraction unit 220 is configured to receive and process an incoming air stream DIA to change the phase state from gaseous to liquid, for at least part of the included moisture. Thereby, EW is extracted from DIA, and an outgoing air stream DOA with reduced humidity is generated. In some embodiments, the water extraction unit 210 is configured to extract EW by direct condensation of the moisture in the incoming air stream, by cooling the air below its dew point, optionally at elevated pressure. For example, the water extraction unit 210 may comprise a conventional evaporator coil, which is arranged to cool DIA, causing water to condense. In these embodiments, box 220A represents the evaporator coil. This type of water extraction is mainly effective when the incoming air has a high relative humidity, such as above approximately 40%. Generally, the quality of EW obtained by this technique is dependent on the quality of the incoming air.

In some embodiments, the water extraction unit 210 is instead configured to extract EW by use of a desiccant. In these embodiments, box 220A represents the desiccant. The desiccant is a hygroscopic substance which is arranged to interact with DIA. During this interaction, the desiccant adsorbs and/or adsorbs water molecules that are present in DIA. The water extraction unit 220 is configured to process the desiccant 220A to release the water molecules, for example by one or more of heating, moisture vapor pressure change, or UV irradiation. The released water molecules are then collected to form EW. This type of water extraction is effective also when the incoming air has a low relative humidity, such as down to 20%, or even lower. Generally, the quality of EW obtained by this technique is dependent on the desiccant, in particular its selectivity towards water.

The DHU 22 in FIG. 1F comprises an air inlet 22A, an air outlet 22B, and a water outlet 22C. The air inlet 22A opens to an air inlet channel 221, which extends to the water extraction unit 220. An air filter 222 is arranged in the air inlet channel 221 for removal of particulate matter, such as debris, dust, etc., and possibly also volatile organic components, carbon, sub-micrometer particles, etc. A pumping device 223 is arranged downstream of the air filter 222, to generate and drive DIA through the water extraction unit 220. A humidity sensor 224 is arranged in the air inlet channel 221 to sense the inlet humidity Hdi of DIA. The air outlet 22B opens to an air outlet channel 225, which extends from the water extraction unit 220. A humidity sensor 226 is arranged in the air outlet channel 225 to sense the outlet humidity Hdo of DOA. The water outlet 22C opens to a water outlet channel 227 which extends to the water extraction unit 220. A flow controller 228 is arranged in the water outlet channel 227 to control the flow of EW from the water extraction unit 220. The flow controller 228 may for example comprise a valve and/or a pumping device. A local control unit 229 is configured to generate control signals for the pumping device 223, the flow controller 228, and the water extraction unit 220, based on sensor data (“measurement signals”) from the humidity sensors 224, 226, to achieve set point data SP2. For example, the set point data SP2 may include a target value for the outlet humidity Hdo and/or a target value for the flow rate of EW (“EW production rate”). The target value for the EW production rate may be set to ensure that a sufficient amount of EW is extracted over a predefined time period, for example 24 hours. The local control unit 229 may, for example, control the flow rate of DIA based on the inlet humidity Hdi to achieve the target value of the EW production rate. Alternatively or additionally, the local control unit 229 may control the flow rate of DIA based on the inlet humidity Hdi to achieve the target value of the outlet humidity Hdo.

Table 1 below shows the required air flow rate, for a given Hdi, to extract 11.1 ml/min of EW, resulting in 16 liters per 24 hours, which corresponds the typical daily consumption in PD therapy. Table 1 also shows the resulting Hdo. As seen in Table 1, the required air flow may become high if Hdi is low and/or if Hdo is to be relatively high. Thus, in certain situations, the DHU 22 may be unable to achieve the target value(s). These situations are mitigated by the provision of the HU 21 and by joint control of the HU 21 and the DHU 22. For example, the HU 21 may be operated to increase Hdi by increasing Hho.

TABLE 1

| Temperature (° C.) | Hdi (%) | Hdo (%) | Required air flow (liters/s) |
|---|---|---|---|
| 20 | 30 | 0 | 37 |
| 20 | 30 | 10 | 55 |
| 20 | 30 | 20 | 110 |
| 25 | 70 | 0 | 11 |
| 25 | 70 | 10 | 13 |
| 25 | 70 | 20 | 16 |
| 25 | 70 | 30 | 19 |
| 25 | 70 | 40 | 26 |
| 25 | 70 | 50 | 39 |
| 25 | 70 | 60 | 78 |

Reverting to FIG. 1C, it is realized that the combination of the HU 21 and the DHU 22 results in a gaseous transportation of tap water TW from the HU 21 to the DHU 22, if the HU 21 and the DHU 22 are arranged so that at least part of HOA from the HU 21 (FIG. 1E) is included in DIA to the DHU 22. This may be achieved by defining a physical channel between the air outlet 21B of the HU 21 and the air inlet 22A of the DHU 22. Alternatively, it may be achieved by arranging the HU 21 to release its outgoing air stream HOA into open air at the location of the HU 21 and by arranging the DHU 22 to receive DIA from open air at the location of the DHU 22, provided that air is allowed to flow from the HU 21 to the DHU 22. The gaseous transportation of water provides various technical advantages.

By the gaseous transportation, the DHU 22 is able to increase its EW production even if the available volume of air is limited, for example when the DHU 22 is located indoors and only operates on indoor air. In the following, the confined space where the HU 21 and DHU 22 are located is generally referred to as “premises” and may be in the home of a patient or in a clinic. The water vapor content of ordinary indoor air is typically 4-15 g per m³ of air for temperatures of 20-25° C. and relative humidity (RH) of 30-70%. Even if the DHU 22 is located in a premises with a high turnover of air, for example through a ventilation system, it may be difficult to provide the required air volume for EW production if the size of the premises is limited. For example, an apartment of 80 m² would require a turnover frequency of about 12 per day to provide a total air volume of 2400 m³. Table 2 below shows the required air volume to extract 16 kg of liquid water for a given air humidity (RH start) at the start of the extraction process. Table 2 also shows the final air humidity (RH end) after the extraction process. As seen in Table 2, the humidity of the indoor air may become undesirably low in order to meet the required air volume, even if the turnover frequency is as high as 12 per day. It is also likely that RH end is even lower, if possible, near the DHU 22. A dry indoor climate may be perceived as unpleasant or even cause or worsen respiratory ailments, as well as cause a sore throat, irritated eyes, dehydration, dry skin, etc. Generally, it may be desirable for the relative humidity in a premises to be in the range of 30-70% or 40-60% for health and comfort.

It may also be noted that EC blood therapy generally requires much larger amounts of dialysis fluid. The required air volume to be processed by the DHU 22 for extraction of EW is correspondingly larger.

TABLE 2

| | | | Required air |
|---|---|---|---|
| Temperature (° C.) | RH start (%) | RH end (%) | volume (m³) |
| 20 | 30 | 0 | 3175 |
| 20 | 30 | 15 | 6349 |
| 25 | 70 | 0 | 960 |
| 25 | 70 | 20 | 1345 |
| 25 | 70 | 40 | 2241 |

The HU 21 and the DHU 22 may be arranged in various configurations to perform the gaseous transportation of tap water. Examples of such configurations are described further below. Typically, all configurations are operable to maintain an acceptable humidity in the premises of the HU 21 and the DHU 22.

Additional technical advantages may also be attained by proper design of the DHU 22. If the DHU 22 is configured to extract EW from a desiccant, it is possible to achieve an inherent purification of EW by use of a desiccant that has a high selectivity towards water. The high selectively implies that the desiccant is tailored to adsorb and/or absorb water molecules rather than other molecules that may be present in DIA. The inherent purification by the DHU 22 may reduce, or even eliminate, the need to process the extracted water by conventional water purification to ensure that TF fulfils regulatory requirements or standards, for example, water for dialysis according to ISO 23500-3. Conventional water purification typically involves Reverse Osmosis (RO), which is quite costly.

In some embodiments, the water extraction unit 220 is configured to produce EW that has conductivity of less than 10 μS/cm, and preferably less than 5 μS/cm or 1 μS/cm. As understood from the foregoing, this may be achieved by using a desiccant with a high selectivity of water.

In some embodiments, the desiccant is an ionic or covalent porous solid, including but not limited to metal-organic and organic porous framework materials, zeolites, organic ionic solids, inorganic ionic solids, organic molecular solids, or inorganic molecular solids, or any combination thereof. The desiccant may be used in a pure, single-phase form, as a composition of different active chemical materials, and/or in combination with performance enhancing additives modulating its properties. Performance enhancing additives may include materials with a high thermal conductivity and molar water absorptivity. The active chemical compound may be used in the form of a powders, extrudates, molded bodies, pressed pellets, pure or composite films, or sintered bodies. In some embodiments, the water capture material comprises an active chemical compound, such as a metal-organic framework (MOF). MOFs are porous materials that have repeating secondary building units (SBUs) connected to organic ligands. In some variations, the SBUs may include one or more metals or metal-containing complexes. In other variations, the organic ligands have acid and/or amine functional group(s). In certain variations, the organic ligands have carboxylic acid groups. Any MOF capable of adsorbing and desorbing water may be employed in the systems provided herein. In some embodiments, MOF-303 is used as desiccant, MOF-303 has a structure of Al(OH)(HPDC), where HPDC stands for 1H-pyrazole-3,5-dicarboxylate. Other conceivable MOFs for use as desiccant include, for example, MOF-801, MOF-841 and MIL-160. A combination of MOFs may also be used as desiccant. Further examples and implementation details are found in the articles “Metal-Organic Frameworks for Water Harvesting from Air”, by Kalmutzki et al., published in Adv. Mater. 2018, 30, 1704304, and “Practical water production from desert air”, by Fathieh et al., published in Sci. Adv. 2018, Vol. 8, Issue 6, which are incorporated herein by reference.

FIG. 2A shows an example of a “non-connected” embodiment, in which the HU 21 and the DHU 22 are physically separated and in fluid communication through open air. The undulated arrow represents the fraction of HOA (FIG. 1E) that reaches the DHU 22 and is included in DIA (FIG. 1F). The non-connected embodiment facilitates installation. Separating the HU 21 from the DHU 22 provides freedom to place the HU 21 near a water tap, for example in a bathroom or kitchen. This makes it easier to supply the HU 21 with tap water, whether achieved by fluidly connecting the HU 21 to the water tap or by manually filling water into a tank associated with the HU 21.

The non-connected embodiment relies on water vapor transport between the HU 21 and the DHU 22, meaning that humid air produced at the HU 21 is transported to the DHU 22 by convection (natural and/or forced) and/or by mixing. This “wireless water transport” between the HU 21 and the DHU 22 through the premises may be more or less effective depending on the transport bandwidth (or, conversely, the flow resistance) between the HU 21 and the DHU 22. The transport bandwidth is dependent on, for example, the distance between the HU 21 and the DHU 22, the ventilation characteristics of the premises, and the presence of obstacles or obstructions such as doors and furniture.

Examples of different installations are shown in FIG. 2B, which is a top plan view of a premises including a bathroom 41, which has a sink 42 and a toilet 43 and is separated by a door 44 from a bedroom 45 with a bed 46. The dialysis therapy is preferably performed while the patient is lying in the bed 46. The therapy system 10′ is therefore located at bedside. As noted above in relation to FIG. 1C, the therapy system 10′ also includes an FGU 24 for generating TF. The DHU 22 is fluidly connected to the therapy system 10′ to provide EW to the FGU 24. The DHU 22 may be placed near the point of treatment to avoid extensive tubing for transfer of EW.

In a first installation example, the HU 21 is placed in the bathroom 41 in fluid connection with a permanent water supply at the sink 21. As long as the door 44 between the bathroom 41 and the bedroom 45 is open, moisture generated by the HU 21 will over time propagate to the DHU 22, for example driven by forced convection. The forced convection may be caused by the pumping device 213 in the HU 21, the pumping device 223 in the DHU 22, or by a separate fan, a ventilation system, etc. In a second installation example, the HU 21 is placed in the down-left corner of the bedroom 45 to be out of the way. The HU 21 may receive tap water from a tank 30, as shown, or through a tubing connected to the water supply in the bathroom 41. When using a tank 30, it may be advantageous for the DHU 22 to be configured to extract more water from the surrounding air than is added by the HU 21. This will reduce the need for tap water and thereby reduce the need for the user to carry tap water from the bathroom 41 to the bedroom 42. In a third installation example, the HU 21 is located close to the DHU 22. Like in the second installation example, the HU 21 may receive tap water from a tank 30, as shown, or through a tubing connected to the water supply in the bathroom 41.

In some embodiments, the non-connected embodiment as shown in FIG. 2A is operable to control the relative humidity in ambient air within the premises. In the illustrated example, a control device 25 is configured to receive sensor data from one or more humidity sensors and provide a target value for the outlet humidity Hho of the HU 21 (FIG. 1E) and a target value for the outlet humidity Hdo of the DHU 22 (FIG. 1F), thereby causing the respective local control unit 219, 229 to operate the HU 21 and the DHU 22 to attain the target values. The target values may be provided as part of the set point data SP1, SP2 in FIG. 2A. The sensor data may include one or more of the inlet humidity Hhi from the sensor 214 in the HU 21, the inlet humidity Hdi from the sensor 224 in the DHU 22, or an ambient humidity Ha from a separate humidity sensor 26 located anywhere within the premises. Although, the sensor 26 is only disclosed for the embodiment in FIG. 2A, it may be present and used in any other embodiment disclosed herein.

The sensor data may be transmitted to the control device 25 by wire or wirelessly. Likewise, the set point data SP1, SP2 may be transmitted by wire or wirelessly to the HU 21 and the DHU 22. The control device 25 may be a separate device, as shown, or may be included one of the local control units 219, 229. Thus, generally, the functionality of the local control units 219, 229 and the control device 25 may be performed by a control arrangement that may be centralized or distributed in any way.

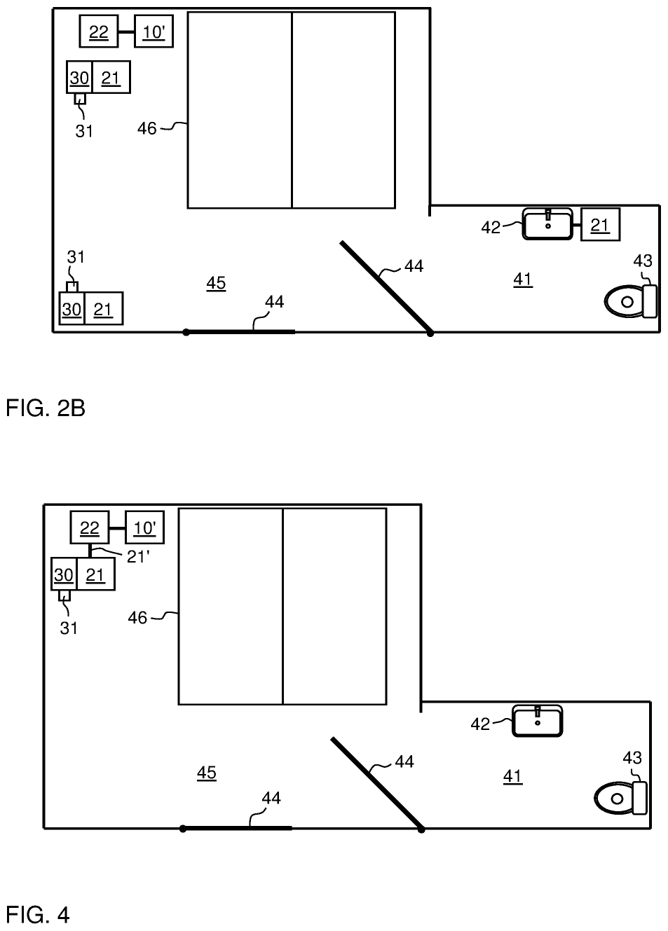

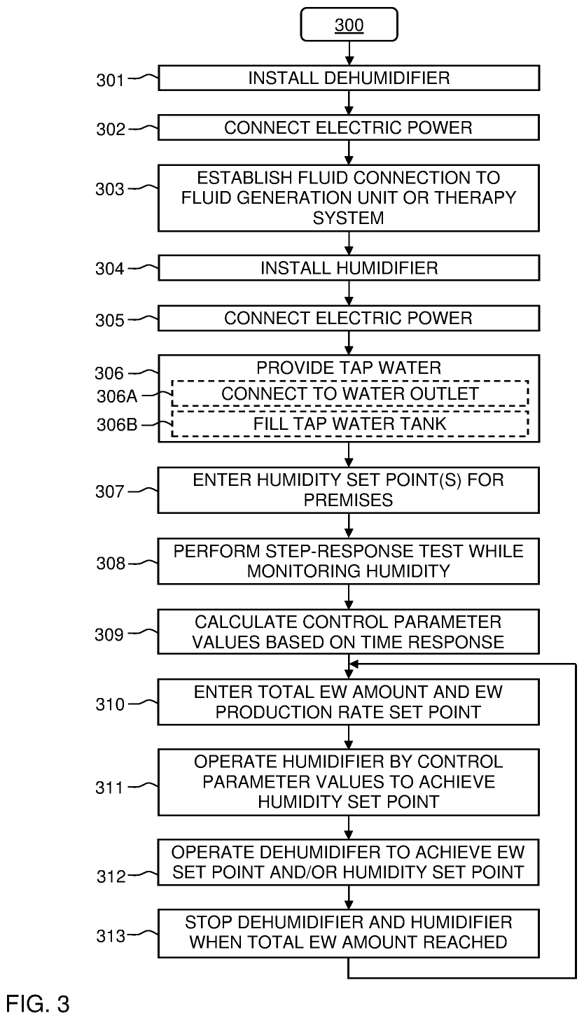

FIG. 3 is a flow chart of an example method 300 of installing and operating the non-connected embodiment as described with reference to FIGS. 2A-2B. In step 301, the DHU 22 is installed at a desired location in the premises, for example close to the FGU 24. In step 302, the DHU 22 is connected to electric power. In step 303, a fluid path for EW is established between the DHU 22 and the FGU 24, or the therapy system 10′ if the FGU 24 is integrated therein (FIG. 1C). For example, tubing may be connected to extend between the water outlet 22C on the DHU 22 and a water inlet on the FGA 24 or the therapy system 10′. In step 304, the HU 21 is installed at a desired location in the premises, for example according to any of the installation examples in FIG. 2B. In step 305, the HU 21 is connected to electric power. In step 306, tap water is provided to the HU 21. For example, the HU 21 may be connected to a permanent water supply (step 306A), or tap water may be filled into a tank associated with the HU 21 (step 306B). In step 307, a user may enter one or more target values for the humidity within the premises (“humidity set point”) into the control arrangement, via an interface device (not shown) such as a keypad, keyboard, touch screen, control buttons, etc. Alternatively, the humidity set point may be automatically set by the control arrangement, for example based on the natural humidity of the premises (when the HU 21 and DHU 22 are stopped), or a humidity determined (by the control arrangement) to provide favorable operating conditions, for example to achieve a net water uptake to minimize the tap water demand.

In step 308, the control arrangement performs a step-response test. The step-response test is performed for the purpose of configuring the control arrangement in relation to the premises. For example, the step-response test may serve to improve the ability of the control arrangement to maintain a suitable humidity within the premises and/or to ensure that sufficient amount of water vapor is received by the DHU 22. Step 308 may be automatically performed whenever the HU 21 and/or DHU 22 is started, or at a predefined time interval, or when an error condition is detected by the control arrangement, for example based on measured humidity. For example, step 308 may be performed when the measured humidity changes in a way that is not expected. Alternatively or additionally, step 308 may be performed on command by the user, for example by the user pressing a dedicated button on the HU 21 or the DHU 22. Alternatively or additionally, the step 308 may be automatically performed to verify input data entered by the user, for example regarding the type or size of the premises.

In the step-response test, the control arrangement causes a step-change in the operating performance of the HU 21, for example in terms of the production rate of water vapor by the HU 21, and/or in the performance of the DHU 22, for example in terms of the consumption rate of water vapor by the DHU 22. In a non-limiting example, the HU 21 may be operated to increase/decrease the flow rate of HOA, and/or to increase/decrease the humidity of HOA. In another non-limiting example, the DHU 22 may be operated to increase/decrease the extraction rate of EW. Generally, the step-change may be generated by changing the operating point of the HU 21 and/or the DHU 22, or by starting/stopping the HU 21 and/or DHU 22. After the step-change, the control arrangement monitors sensor data representative of the humidity in the premises over time. For example, the sensor data may represent one or more of Hdi, Hhi, Ha (FIG. 2A). The time response in the sensor data will be representative of the above-mentioned transport bandwidth of the premises. In step 309, the control arrangement determines suitable control parameter values for the joint control of the HU 21 and the DHU 22, based on the time response in the sensor data, for example from a look-up table or by evaluation of one of more predetermined functions in accordance with well-known control theory. Depending on the type of controller implemented by the control arrangement, the control parameters may relate to one or more of Gain (P), Integral (I) or Derivative (D).

In step 310, the user may input further control data to the control arrangement, including a set point (target value) for the EW production rate by the DHU 22, and optionally a total amount of EW (“total EW amount”) to be extracted by the DHU 22 for a given operating period. Alternatively, the control data may be retrieved by the control arrangement from a memory unit in the dialysis system, for example in the therapy system 10, the FGU 24, the HU 21 or the DHU 22. In step 311, the control arrangement operates the HU 21 by use of the control parameter values from step 309, to achieve the humidity set point (entered in step 307). In step 312, the control arrangement operates the DHU 22 by use of the control parameter values from step 309, to achieve the humidity set point and/or to achieve the set point for the EW production rate (entered in step 310). It may be noted that HU 21 and DHU 22 may be jointly operated towards plural humidity set points, given by different humidity sensors. It is also conceivable that HU 21 and DHU 22 are separately operated towards different humidity set points, given by different humidity sensors. It should also be understood that, for a time period after start-up of the HU 12 and the DHU 22, humidity will be unevenly distributed between the HU 21 and the DHU 22 until a steady-state condition is reached.

In step 313, the HU 21 and the DHU 22 are stopped when an accumulated amount of EW exceeds a threshold value, which may be the total EW amount entered in step 310 or a predefined value. The accumulated EW amount may be determined in any suitable way, such as by flow measurement, volumetric metering, etc. for example by the flow controller 228 (FIG. 1F) or a separate device. In an alternative, the accumulated amount may be given by measuring the fluid level in a tank for collection of EW (cf. storage unit 23 in FIG. 1C), or by measuring the weight of such a tank. A sensor for such measurement is schematically represented as 23″ in FIG. 1C. As shown by an arrow, the method 300 may then return to perform step 310 when the HU 21 and the DHU 22 are re-started, for example in accordance with a predefined schedule or by user command. Alternatively, as indicated above, the method 300 may return to step 308 and perform the step-response test when the HU 21 and the DHU 22 are re-started.

It is conceivable that at least one of the HU 21 and the DHU 22 comprises a positioning device, which is configured to generate position data indicative of the relative positioning of the HU 21 and the DHU 22. The relative positioning may supplement or replace the step-response test (step 308) and be used to estimate the transport bandwidth between the HU 21 and the DHU 22 and/or to determine the control parameters (step 309). In one example, the positioning device is configured to measure the distance between the HU 21 and the DHU 22, by any conceivable technique. If data is exchanged wirelessly between the HU 21 and the DHU 22, the distance may be estimated based on signal strength. In other examples, more advanced positioning is used, for example by connecting the HU 21 and the DHU 22 to an indoor positioning system. In a further alternative, the user may input the distance via the interface device, for use by the control arrangement.

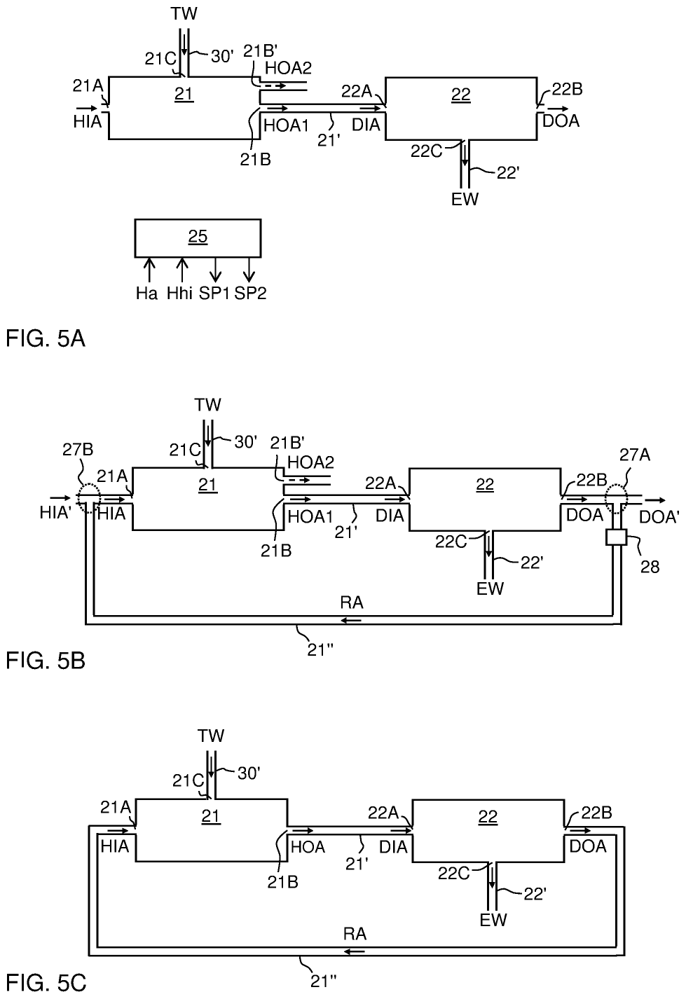

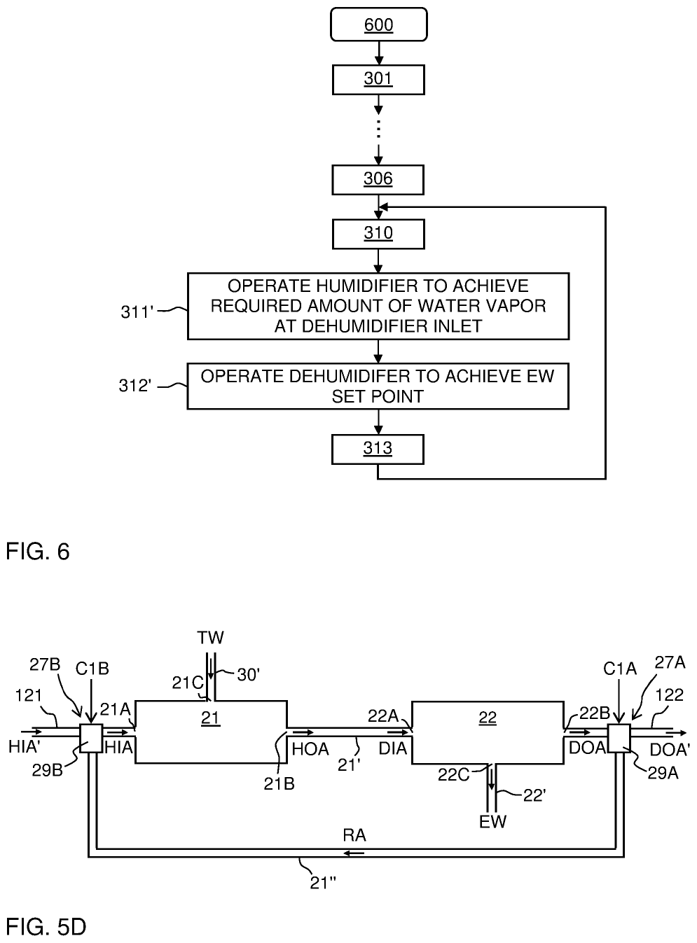

FIGS. 5A-5C show various examples of a “connected” embodiment, in which at least one fluid connecting device, FCD 21′, 21″ is arranged to extend between the HU 21 and the DHU 22. The FCD 21′, 21″ defines at least one fluid channel. The FCD 21′ is arranged to fluidly connect the air outlet 21B of the HU 21 with the air inlet 22A of the DHU 22, to thereby include at least part of the outgoing air stream from the HU 21 in the incoming air stream to the DHU 22. In the examples of FIGS. 5B-5C, an additional FCD 21″ is arranged to fluidly connect the air outlet 22B of the DHU 22 with the air inlet 21A of the HU 21, to thereby recirculate at least part of DOA for inclusion in HIA. One general advantage of the connected embodiment over the non-connected embodiment is a more efficient and direct control of the humidity of DIA, by eliminating the impact of the transport bandwidth of the premises.

FIG. 4 shows an installation example of the connected embodiment in the premises of FIG. 2B. The DHU 22 is located close to the therapy system 10′, which is arranged at bedside. The HU 21 is fluidly connected to the DHU 22 by the FCD 21′ (and optionally the FCD 21″) and arranged in the vicinity of the DHU 22 to reduce the extent of the FCD 21′. Although not shown in FIG. 4, it is conceivable that the HU 21 and DHU 22 are combined into a single unitary structure. The unitary structure may be space-efficient and facilitate installation, handling and control. The HU 21 may receive tap water from a tank 30, as shown, or through a tubing connected to the water supply in the bathroom 41. In other installation examples, not shown, the HU 21 may be spaced from the DHU 22, for example as shown in FIG. 2B, with the FCD 21′ (and optionally the FCD 21″) extending between the HU 21 and the DHU 22.

Except for the FCD 21′, the example in FIG. 5A is similar to the non-connected embodiment in FIG. 2A and may be configured and operated similarly. In the HU 21, TW is received at water inlet 21C, HIA is received at air inlet 21A, and an outgoing air stream HOA1 is emitted at air outlet 21B, which is connected to the FCD 21′. As shown in FIG. 5A, the HU 21 may define another air outlet 21B′, which emits another outgoing air stream HOA2 into the surroundings of the HU 21. The air streams HOA1 and HOA2 may be diverted from a common outgoing air stream (cf. HOA in FIG. 1E) or be two independently generated air streams. The air outlet 21B′ and HOA2 are optional. In the DHU 22, DIA is effectively identical to HOA1 and received from the FCD 21′ at air inlet 22A, DOA is emitted at air outlet 22B, and EW is provided at water outlet 22C. A control device 25 is configured to control the combination of HU 21 and DHU 22. The control device 25 may be configured in correspondence with the control device 25 in FIG. 2A, As noted above, the functionality of the local control units 219, 229 (FIGS. 1E-1F) and the control device 25 may be generally incorporated into a control arrangement that may be centralized or distributed in any way.

The provision of HOA2 may improve the ability of the control arrangement to control the relative humidity in ambient air within the premises. The control arrangement may control the outlet humidity Hdo of DOA and/or the outlet humidity of HOA2 to attain a target humidity in the premises, as given by Hdi from sensor 224 (FIG. 1F), or Ha from a separate humidity sensor (cf. 26 in FIG. 2A). It is realized that if HOA1 and HOA2 are independently generated by the HU 21, the control arrangement may operate on a separate humidity value for HOA2, given by an additional humidity sensor (not shown). Otherwise, the outlet humidity Hho from sensor 216 (FIG. 1E) represents both HOA1 and HOA2.

The example in FIG. 5B is an “open recirculation system”, in which the FCD 21″ is installed to recirculate a portion of DOA. The FCD 21″ is a structural recirculation device that defines at least one fluid channel for recirculated air, RA. The FCD 21″ extends from a junction 27A downstream of the water extraction unit (220 in FIG. 1F) to a junction 27B upstream of the evaporation unit (210 in FIG. 1E). The junctions 27A, 27B may be located externally (as shown) or internally of the HU 21, DHU 22. The remainder of DOA, designated DOA′, is emitted to the surroundings of the DHU 22. RA is directed by the FCD 21″ to the air inlet 21A and forms part of HIA, which also includes an incoming air stream HIA′ from the surroundings of the HU 21. In the open recirculation system, a fraction of the air that is processed by the HU 21 and the DHU 22 is continuously replaced. Thereby, like the configurations in FIG. 2A and FIG. 5A, the open recirculation system may be operated to extract a net amount of water vapor from the ambient air, to reduce the consumption of TW. The open recirculation system may also be operated, like the system in FIG. 5A, to control the relative humidity in ambient air within the premises, by controlling the outlet humidity Hdo of DOA and/or the outlet humidity of HOA2, to attain a humidity set point for the ambient air in the premises. However, if the humidity in the premises is given by Hhi, the sensor 214 (FIG. 1E) may be moved, or supplemented by an additional sensor, to measure the humidity of HIA′ rather than HIA. If the HU 21 and the DHU 22 is combined into a unitary structure, it is conceivable that DOA′ and HOA2 are mixed and that the humidity of the resulting mixture is measured by a dedicated humidity sensor, as an alternative or supplement to separately measuring the humidity of DOA and HOA2.

As shown in FIG. 5B, a flow controller 28 may be arranged in the FCD 21″ to set the fraction of DOA to be diverted into the FCD 21″, denoted “recirculation fraction” in the following. The flow controller 28 may be a valve, pumping device or the like. The amount of RA is thereby adjustable, for example by a control signal from the control arrangement. The recirculation fraction determines the degree of air exchange with the environment and may either be set by the user or by the control arrangement, for example based on sensor data, based on the step-response test, or a combination thereof. The DHU 22 may require a certain combination of air flow rate and humidity from the HU 21 to be able to reach the EW production target. The control arrangement may thus operate the HU 21 accordingly.

The method 300 in FIG. 3 is equally applicable to the connected embodiments shown in FIGS. 5A-5B although some steps may be modified, as outlined hereinabove, in view of the differences in structure. Further, in respect of the open recirculation system in FIG. 5B, the method 300 may comprise a step of determining a fraction of DOA to be recirculated, and a step of operating the system to achieve this fraction.

The system in FIG. 5B has potential advantages over the system in FIG. 5A. By the recirculation, the flow rate of air from and to the surroundings may be reduced, resulting in more silent operation. Also, the system in FIG. 5B may be easier to control, since it is less dependent on the properties of the surroundings.

The example in FIG. 5C is a “closed recirculation system”, in which the FCD 21″ is installed to recirculate air through the HU 21 and the DHU 22 without air exchange with the surroundings. Specifically, the FCD 21″ connects the air outlet 22B with the air inlet 21A, so that DOA forms HIA. The air is thus used as an internal medium for water vapor transport from the HU 21 to the DHU 22. This also means that the consumption of TW in the HU 21 is effectively equal to the EW production rate in the DHU 22.

FIG. 6 is a flow chart of an example method 600 for installing and operating a closed recirculation system as shown in FIG. 5C. The manual installation steps 301-306 may be the same as in the method 300 (FIG. 3). However, compared to the method 300, steps 307-309 are omitted. After the manual installation, the method 600 comprises step 310, which may be the same as in the method 300. Thus, in step 310, the user may input further control data to the control arrangement, including a set point for the EW production rate by the DHU 22, and optionally a total EW amount to be extracted by the DHU 22 for a given operating period. Alternatively, the control data may be retrieved by the control arrangement from a memory unit in the dialysis system, for example in the therapy system 10, the FGU 24, the HU 21 or the DHU 22. In steps 311′-312′, the HU 21 is operated to achieve a required amount of water vapor at the inlet 22A of the DHU 22, and the DHU 22 is operated to meet the set point for the EW production rate. The amount of water vapor at the inlet 22A is governed by a combination of the flow rate and the humidity of HOA. The control arrangement may determine a combination of flow rate and humidity from the HU 21 that allows the DHU 22 to reach the set point of EW production rate, and operate the HU 21 accordingly. For example, a suitable operating point for the HU 21 and the DHU 22 may be determined based on data corresponding to Table 1 (above). For example, according to Table 1, if HU 21 is operated to generate HOA with a humidity of 70%, and the DHU 22 is operated to generate DOA with a humidity of 20%, the required air flow rate of HOA is 16 liters/second, if the temperature is 25° C. and the total EW amount is 16 liters per 24 hours. Like in the method 300, the method 600 comprises a step 313, which stops the HU 21 and the DHU 22 when the accumulated amount of EW exceeds a threshold value. The method 600 may then return to perform step 310 when the HU 21 and the DHU 22 are re-started.

Compared to other variants, the closed recirculation system maximizes the humidity differences over the HU 21 and DHU 22 and thereby minimizes the air flow rate through them. This also enables downsizing of the system.

Another potential advantage of the closed recirculation system, which repeatedly humidifies and dehumidifies the same air, is that volatile contaminants initially present in the air will be removed early in the process if the water extraction unit 220 (FIG. 1F) is unable to purify the extracted water EW. Such a water extraction unit 220 may be based on direct condensation. The volatile contaminants will be contained in the initial portion of EW. By operating the system to discard the initial portion of EW, the impact of air impurity may be mitigated or even eliminated.

It may be noted that the closed recirculation system in FIG. 5C need not circulate air through HU 21 and DHU 22. For example, air may be replaced by any gas that is deemed to improve the performance and/or durability of the system.

In some embodiments, the system is configured to be switched, manually or by the control arrangement, between operating modes corresponding to two or more of the configurations in FIG. 5A, FIG. 5B and FIG. 5C. An example of such a system is shown in FIG. 5D. The FCD 21″ is arranged to fluidly connect an outlet line 122 and an inlet line 121 of the system. In the illustrated example, the outlet line 122 extends from the air outlet 22B on the DHU 22 to the surroundings, and the inlet line 121 extends from the surroundings to the air inlet 21A on the HU 21. The FCD 21″ is fluidly connected to the outlet line 122 at outlet junction 27A and to the inlet line 121 at inlet junction 27B. In the illustrated example, a respective three-way valve 29A, 29B is arranged at each of the junctions 27A, 27B. The valves 29A, 29B may be seen to form a flow controller that is operable to selectively switch the system between operating modes. In FIG. 5D, the flow controller 29A, 29B may include the function of the flow controller 28 in FIG. 5B. The valve 29B at the inlet junction 27B is optional but may be included for better control of the admission of ambient air into the HU 21. It may be noted that the system is operable in all modes even if the valve 29B is omitted. The provision of the valve 29B may reduce noise in the closed mode.

The flow controller 29A, 29B is operable to control the flow rate of RA through the FCD 21″, and thus the recirculation fraction. The flow controller 29A, 29B may include any number of valves of any type, one or more pumps, one or more flow restrictors, etc.

The system in FIG. 5D may be operated in an open mode without recirculation (cf. FIG. 5A), an open mode with recirculation (cf. FIG. 5B), or a closed mode with recirculation (cf. FIG. 5C). The choice of operating mode may be based on the size of the premises, the air humidity in the premises, or user preferences. The operating mode may be changed during operation of the system. In a preferred embodiment, selection of operating mode is made by the control arrangement, by generating a control signal for the flow controller 29A, 29B.

The flow controller 29A, 29B may be operated to set the recirculation fraction to any value from 0% (open mode without recirculation) to 100% (closed mode). In some embodiments, the recirculation fraction may be set to one or more predefined values between 0% and 100% to operate the system in the open mode with recirculation. Alternatively, the recirculation fraction may be changed continuously by the valve arrangement 29A, 29B between 0% and 100%, or in any other range. In some embodiments, the system is switched between the closed mode and one of the open modes, for example the open mode without recirculation. This corresponds to toggling the flow controller 29A; 29B between a recirculation fraction of 0% and a recirculation fraction of 100%.

The closed mode may be selected to minimize the required air flow rate and/or to avoid affecting the ambient air in the premises. On the other hand, the open modes enable air exchange with the environment and offer an opportunity of decreasing the tap water demand by extracting a fraction of the water from the environment instead of from vaporized tap water. Furthermore, the open modes enable control of the air humidity in the premises.

Another reason for switching between operating modes may be to reduce the noise of the system. The closed mode is likely to be more silent than the open modes and may thus be used during night, whereas an open mode may be used during the day, for example to also extract water from the environment.

Although not shown in FIG. 5D, the system may also be operable in a pure DHU mode, in which the HU 21 is disabled. In the pure DHU mode, the system may be configured to pass ambient air through the disabled HU 21 to the inlet 22A of the DHU 22. Alternatively, DIA may be admitted into the DHU 22 through an air inlet valve (not shown) on the FCD 21′ or the DHU 22. The control arrangement may set the system in the pure DHU mode when the ambient air and the premises are found to support sufficient water extraction. For example, the pure DHU mode may be used in premises in a climate with high moisture content, especially when the premises admit outdoor air, for example through an open door or window. In another example, the pure DHU mode may be used in a premises with a high turnover of air, for example through an air conditioning system. The control arrangement may detect any such premises by the step-response test, based on information entered by the user, or a combination thereof.

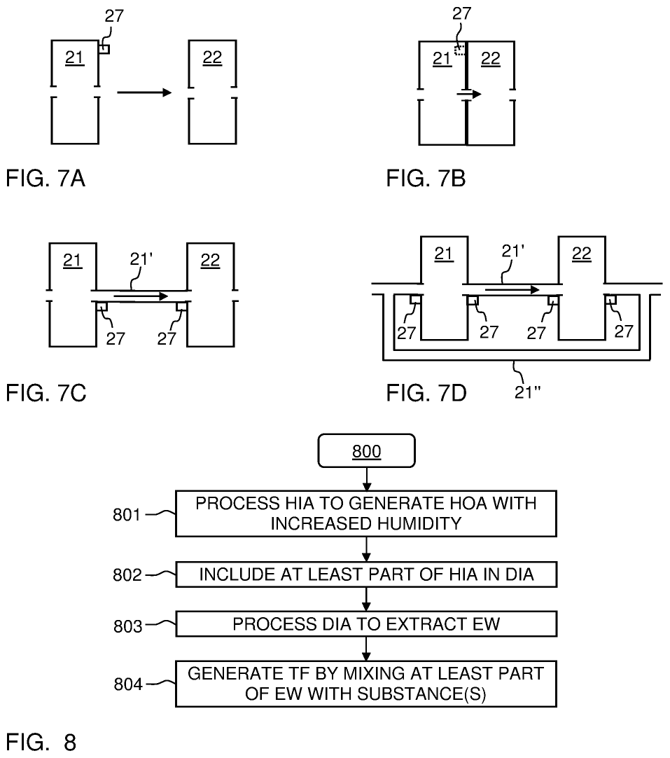

It is conceivable that the HU 21 and the DHU 22 are configured as modules that may be structurally combined to form a non-connected embodiment or a connected embodiment of any type. Such a modular system may comprise a connection sensor, which is configured to sense a fluid connection state between the HU 21 and the DHU 22. The control arrangement may be responsive to the fluid connection state indicated by the connection sensor and modify its operation accordingly. Depending on fluid connection state, the control arrangement may select different operating parameters and/or selectively include or exclude operation steps. Some non-limiting examples of the use of connection sensors are shown in FIGS. 7A-7D. In FIG. 7A, HU 21 and DHU 22 are spaced apart in a non-connected embodiment, and a connection sensor 27 indicates the spaced-apart state. In FIG. 7B, HU 21 and DHU 22 are brought into mutual engagement to define a confined fluid channel for the humidified air stream (HOA), and the connection sensor 27 indicates an open state without recirculation. In FIG. 7C, HU 21 and DHU 22 are connected by a tubing 21′, and connection sensors 27 indicate an open state without recirculation. In FIG. 7D, HU 21 and DHU 22 are connected by both tubing 21′ and recirculation tubing 21″, and connection sensors 27 indicate an open state with recirculation. In the illustrated examples, the connection sensor(s) 27 are automatically activated depending on how HU 21 and DHU 22 are connected. The respective connection device 27 in FIGS. 7A-7D may be of any type, for example a mechanical switch, a magnetic sensor, a capacitive sensor, etc. In a variant, a connection device 27 is manually activated by the user to indicate a fluid connection state to the control arrangement.

In a variant of the non-connected embodiment (FIG. 2A), the DHU 22 comprises an auxiliary humidifier, which may be operated to supplement the HU 21 if the transport bandwidth is insufficient, for example as a result of a large distance between the HU 21 and the DHU 22, poor ventilation in the premises, or temporary blockage of the passage from the HU 21 to the DHU 22. The need to operate the auxiliary humidifier may be set by the user or be inferred by the control arrangement based on the step-response test and/or the distance (if known). For example, the user may prefer to place the HU 21 in the bathroom close to the water supply. If the transport bandwidth is limited, the user may fill a first amount of tap water into a tank at the HU 21 and a second amount of tap water into a tank at the DHU 22. The second amount may be smaller than the first amount if the HU 21 is operated as the main humidifier in the premises.

It is conceivable to connect the HU 21 to receive HIA from outdoor air and/or to connect the DHU 22 to provide DOA to outdoor air. This reduces the impact of the system on the humidity within the premises. It may also reduce the required capacity of the HU 21.

Reverting to the installation examples in FIG. 2B and FIG. 4, it may be advantageous to be able to provide an advance notification to the user that the tank 30 needs to be refilled, and possibly the amount of water that needs to be added to the tank 30 to facilitate for the user. The advance notification may be given through a feedback unit connected to the control arrangement, for example a display or a speaker. In one embodiment, as indicated in FIG. 2B and FIG. 4, the tank 30 comprises a level sensor 31 that generates a signal indicative of the current fill level of the tank 30. The level sensor 31 may be of any conventional type, for example ultrasonic, capacitive, optical, magnetostrictive, magnetoresistive, etc. Alternatively, the level sensor 31 may measure the weight of the tank 30. The control arrangement may determine a TW consumption rate based on the signal from the level sensor 31 and estimate, by prediction or extrapolation, a time point when the tank 30 will be empty. The control arrangement may then signal to the user, well in advance of this time point, a need to replenish the tank 30 and, possibly, the amount of water to be added to the tank. In another embodiment, the control arrangement is configured to estimate the TW consumption rate based on sensor data for the humidity in the premises. The sensor data may include one or more of the inlet humidity Hhi (sensor 214 in FIG. 1E), the inlet humidity Hdi (224 in FIG. 1F), or the ambient humidity Ha (sensor 26 in FIG. 2A). The TW consumption rate may be estimated by use of a calculation model of the system as connected to a therapy system 10, 10′. The calculation model may be based on analytical functions or be a machine learning-based model. The calculation model is configured to account for the water consumption rate by the therapy system through its use of treatment fluid. The consumption rate of treatment fluid may be given by a therapy prescription or measured. The calculation model is configured to estimate the system's production rate of water, EW, from ambient air. The EW production rate depends on the settings of the system, which are known, and the humidity of the ambient air over time, which is given by or predicted from the sensor data. The calculation model may take into account the outcome of one or more step-response tests to predict the humidity. The TW consumption rate is given by the difference between the EW production rate and the water consumption rate of the therapy system. Given the calculation model, the time point when the tank 30 will be empty may be estimated. The control arrangement may signal to the user, well in advance of this time point, a need to replenish the tank 30 and, possibly, the amount of water to be added to the tank.

FIG. 8 is a flow chart of a method 800 of providing treatment fluid, which may be performed by the systems disclosed herein. In step 801, an input air stream, HIA, is processed to generate an output air stream, HOA, with increased humidity compared to the input air stream, HIA. Step 801 may be performed by the HU 21. In step 802, at least part of the output air stream, HOA, is included in a further input air stream, DIA, to be processed in step 803. Step 802 may be performed by arranging the HU 21 in relation to an DHU 22, with or without use of a fluid connecting device 21′. In step 803, the further input air stream, DIA, is processed for extraction of liquid water, EW. Step 803 may be performed by the DHU 22. In step 804, treatment fluid is generated by mixing at least part of the liquid water, EW, with one or more substances. Step 804 may be performed by the FGU 24.

It should be understood that the control arrangement may operate on either relative humidity or absolute humidity. For example, by also measuring temperature, a measured relative humidity may be converted into an absolute humidity.

While the subject of the present disclosure has been described in connection with what is presently considered to be the most practical embodiments, it is to be understood that the subject of the present disclosure is not to be limited to the disclosed embodiments, but on the contrary, is intended to cover various modifications and equivalent arrangements included within the spirit and the scope of the appended claims.

Further, while operations are depicted in the drawings in a particular order, this should not be understood as requiring that such operations be performed in the particular order shown or in sequential order, or that all illustrated operations be performed, to achieve desirable results.

1-26. (canceled)

27. A system for providing treatment fluid for dialysis therapy, the system comprising:
a fluid generation unit configured to generate the treatment fluid by mixing water with one or more substances,
a dehumidifier unit configured to receive a first stream of air, process the first stream of air for extraction of liquid water, and provide the liquid water for the fluid generation unit,
a humidifier unit configured to receive a second stream of air, and process the second stream of air to generate a third stream of air with increased humidity compared to the second stream of air, wherein the humidifier unit is arranged to include at least part of the third stream of air in the first stream of air,
at least one fluid connecting device between the humidifier unit and the dehumidifier unit,
the humidifier unit is configured to emit at least part of the third stream of air into the at least one fluid connecting device, and the dehumidifier unit is configured to receive the first stream of air from the at least one fluid connecting device,
the dehumidifier unit is configured to output a fourth stream of air which is generated when the first stream of air is processed for extraction of the liquid water, and
the system further defines a fluid channel, which is configured to re-direct at least part of the fourth stream of air to an air inlet of the humidifier unit, to include said at least part of the fourth stream of air in the first stream of air.

28. The system of claim 27, wherein the dehumidifier unit comprises a desiccant, which is arranged to adsorb and/or absorb moisture from the first stream of air and which is processed by the dehumidifier unit to extract the liquid water from the desiccant.

29. The system of claim 28, wherein the desiccant is configured to have a high selectivity towards water.

30. The system of claim 27, wherein the liquid water that is extracted from the first stream of air has conductivity of less than 10 μS/cm, and preferably less than 5 μS/cm or 1 μS/cm.

31. The system of claim 27, further comprising a control arrangement, which is configured to jointly operate the humidifier unit and the dehumidifier unit to achieve a set point that defines air humidity within a space that comprises the humidifier unit and the dehumidifier unit.

32. The system of claim 31, wherein the control arrangement is further configured to operate the dehumidifier unit to achieve a set point that defines an amount of liquid water extracted per unit time.

33. The system of claim 31, wherein the control arrangement is further configured to deactivate the humidifier unit and the dehumidifier unit when an accumulated amount of liquid water exceeds a threshold value.

34. The system of claim 27, further comprising at least one humidity sensor, which is arranged to generate a measurement signal representing the air humidity.

35. The system of claim 27, wherein the humidifier unit and the dehumidifier unit are combined into a unitary structure.

36. The system of claim 27, wherein the fluid channel is arranged to redirect the fourth stream of air to the air inlet of the humidifier unit, so that the fourth stream of air forms the first stream of air.

37. The system of claim 27, which comprises a flow controller which is operable to set how much of the fourth stream of air that is re-directed to the air inlet of the humidifier unit.

38. The system of claim 37, which, by operation of the flow controller, is switchable between a first operating mode and a second operating mode, wherein the system, in the first operating mode, is configured to admit surrounding air into the second stream of air and provide at least part of the fourth stream of air into the surrounding air, and wherein the system, in the second operating mode, is configured to direct the fourth stream of air to the air inlet of the humidifier unit, so that the fourth stream of air forms the first stream of air.

39. The system of claim 38, which, in the first operating mode, is configured to provide all of the fourth stream of air into the surrounding air.

40. The system of claim 37, which comprises an inlet line, which is connected to the air inlet of the humidifier unit, and an outlet line, which is connected to an outlet of the dehumidifier unit to receive the fourth stream of air from the dehumidifier unit, wherein the fluid channel is fluidly connected at a first junction on the outlet line and at a second junction on the inlet line, and wherein the flow controller is arranged to control an air flow rate through the fluid channel from the outlet line to the inlet line.

41. The system of claim 27, wherein the humidifier unit is configured to receive tap water and process the tap water to generate the third stream of air from the second stream of air and the tap water.

42. The system of claim 41, wherein the humidifier unit is connected to receive the tap water from a tank, and wherein the system is configured to estimate a time point of depletion of the tank, based on a signal from a level sensor of the tank or based on measured air humidity within a space that comprises the humidifier unit and the dehumidifier unit, and output, in advance of said time point, a user instruction to add tap water to the tank.

43. The system of claim 42, which is configured to estimate the time point of depletion by use of a calculation model, which is configured to predict the extraction of the liquid water as a function of air humidity, and by further use of data indicative of consumption of the treatment fluid by an operating therapy system, which is connected to receive the treatment fluid from the system.

44. The system of claim 27, further comprising a storage unit, which is arranged to receive and accumulate the liquid water from the dehumidifier unit or the treatment fluid from the fluid generation unit.

45. A computer-implemented method of operating the system as defined in claim 27, the method comprising: receiving at least one measurement signal representing the air humidity within a space that comprises the humidifier unit and the dehumidifier unit, and jointly operating the humidifier unit and the dehumidifier unit to achieve a predefined air humidity within the space as indicated by the at least one measurement signal.
